(19) United States
(12) Patent Application Publication
(10) Pub. No.: US 2013/0210638 A1
(43) Pub. Date: Aug. 15, 2013

(54) METHODS FOR SEQUENCING NUCLEIC ACID

(71) Applicants: Jeffrey Charles Olson, Chelmsford, MA (US); James Brayer, Windham, NH (US); Andrew Watson, Bedford, MA (US)

(72) Inventors: Jeffrey Charles Olson, Chelmsford, MA (US); James Brayer, Windham, NH (US); Andrew Watson, Bedford, MA (US)

(21) Appl. No.: 13/762,843

(22) Filed: Feb. 8, 2013

Related U.S. Application Data
(60) Provisional application No. 61/597,604, filed on Feb. 10, 2012.

(51) Int. Cl. C12Q 1/68

(52) U.S. Cl. CPC C12Q 1/6869
USPC 506/2

(57) ABSTRACT

The invention generally relates to methods for sequencing a nucleic acid template from both a 5′ and a 3′ end in a single sequencing reaction. In certain embodiments, methods of the invention involve amplifying a nucleic acid template to produce a plurality of amplicons, splitting the amplicons into first and second portions, attaching a first oligonucleotide including a first universal primer site to a 5′ end of the amplicons in the first portion, and attaching a second oligonucleotide including a second universal primer site to a 3′ end of the amplicons in the second portion. The first and second primer sites may be the same or may be different. The method additionally involves pooling the first and second portions, and sequencing the pooled amplicons, thereby sequencing a nucleic acid template from both a 5′ and a 3′ end in a single sequencing reaction.

### RELATED APPLICATION

The present application claims the benefit of and priority to U.S. provisional application Ser. No. 61/597,604, filed Feb. 10, 2012, the content of which is incorporated by reference herein in its entirety.

### FIELD OF THE INVENTION

The invention generally relates to methods for sequencing a nucleic acid template from both a 5′ and a 3′ end in a single sequencing reaction.

### BACKGROUND

Modern developments in DNA sequencing technologies have allowed for the rapid determination of nucleic acid sequences, from targeted genomic regions to full genomic analysis, rapidly accelerating advances in medicine and research. Such advances are due largely to the advent of next-generation sequencing platforms that do not rely on gel-based separation of sequence fragments produced by Sanger dideoxy sequencing methods.

Briefly, a single-stranded nucleic acid (e.g., DNA or cDNA) is hybridized to oligonucleotides attached to a surface of a flow cell. The single-stranded nucleic acids may be captured by methods known in the art. The oligonucleotides may be covalently attached to the surface or various attachments other than covalent linking as known to those of ordinary skill in the art may be employed. Moreover, the attachment may be indirect, e.g., via the polymerases of the invention directly or indirectly attached to the surface. The surface may be planar or otherwise, and/or may be porous or non-porous, or any other type of surface known to those of ordinary skill to be suitable for attachment. The nucleic acid is then sequenced by imaging the polymerase-mediated addition of fluorescently-labeled nucleotides incorporated into the growing strand surface oligonucleotide, at single molecule resolution. Such methods allow for many nucleic acid templates to be sequenced at any given time in a single sequencing reaction. Such sequencing methods are generally limited to sequencing a single nucleic acid template in only one direction. Generally, the template is attached to the solid support at the 3′ end and sequencing-by-synthesis occurs in the 5′ to 3′ direction, away from the surface of the solid support. Sequencing a template in only a single direction limits the amount of information that can be obtained from any nucleic acid template. For example, the average read lengths on next generation sequencing platforms is as follows: Roche/454, 250 bases; Illumina/Solexa, 25 bases; SOLiD, 35 bases; Heliscope, 25 bases. Such reads are not optimal for the de novo assembly of genomes. Additionally, the detection and proper placement of amplifications, inversions, and translocations using these short reads are severely limited. The proper detection and placement of short indels are also difficult. Short reads may therefore be problematic when sequencing highly polymorphic or highly aberrant genomes. For example, the occurrence of Large-scale Copy-number Variations (LCVs) in normal (non-disease) individuals is an indication that acquiring an accurate description of human genetic variation may require more than the detection of single-nucleotide polymorphisms. Genetic rearrangements are even more heterogeneous and prevalent in cancer genomes, underscoring the importance of their proper detection and characterization.

In addition to the above limitations of short reads, another inherent disadvantage of single molecule sequencing technologies is a high per-read error rate. This is due to the all-or-none signal detection during an incorporation event and the increased susceptibility to contaminating nucleotides. For instance, the incorporation of an unlabeled nucleotide contaminant in a single nascent strand of complementary DNA will produce a failed detection event or a deletion in the read relative to the reference. Sequencing errors in short reads are especially problematic as they complicate proper alignment of the reads onto a reference sequence.

### SUMMARY

The invention provides methods that allow for sequencing a nucleic acid template in a 5′ and in a 3′ direction in a single sequencing reaction, i.e., bi-directional sequencing. This approach allows for improved coverage of an entire nucleic acid template as well as an ability to sequence both the 5′ and 3′ strand. Methods of the invention are particularly useful for sequencing longer templates and can be used to help assemble regions of the genome that would otherwise be difficult to assemble due to repeats or homology to other regions. Methods of the invention also allow for coverage of bases from both directions, which can increase accuracy of base calls, especially if the quality score decreases with read length.

In certain aspects, methods of the invention involve amplifying a nucleic acid template to produce a plurality of amplicons, splitting the amplicons into first and second portions, attaching a first oligonucleotide including a first universal primer site to a 5′ end of the amplicons in the first portion, and attaching a second oligonucleotide including a second universal primer site to a 3′ end of the amplicons in the second portion. The first and second primer sites may be the same or they may be are different. The method additionally involves pooling the first and second portions, and sequencing the pooled amplicons, thereby sequencing a nucleic acid template from both a 5′ and a 3′ end in a single sequencing reaction.

Any amplification method known in the art can be used with methods of the invention. Exemplary amplification methods include, but are not limited to, rolling circle amplification, displacement replication, cloning, the polymerase chain reaction (PCR) and variations of the PCR method including, but not limited to, qPCR, multiplex PCR, asymmetric PCR, nested PCR, hotstart PCR, touchdown PCR, assembly PCR, digital PCR, allele specific PCR, methylation specific PCR, reverse transcription PCR, helicase dependent PCR, inverse PCR, intersequence specific PCR, ligation mediated PCR, mini primer PCR, and solid phase PCR, emulsion PCR, and PCR as performed in a thermocycler, droplets, microfluidic reaction chambers, flow cells and other microfluidic devices.

Methods of the invention involve splitting the amplicons into first and second portions. Any technique known in the art may be used to split the amplicons into first and second portions. Exemplary compartmentalizing techniques are shown for example in, Griffiths et al. (U.S. Pat. No. 7,968,287) and Link et al. (U.S. patent application number 2008/0014589), the content of each of which is incorporated by reference herein in its entirety. In certain embodiments, the compartmentalizing involves forming droplets and the compartmentalized portions are the droplets. An exemplary method involves for forming droplets involves flowing a stream of sample fluid including the amplicons such that it intersects two opposing streams of flowing carrier fluid. The carrier fluid is immiscible with the sample fluid. Intersection of the sample fluid with the two opposing streams of flowing carrier fluid results in partitioning of the sample fluid into individual sample droplets. The carrier fluid may be any fluid that is immiscible with the sample fluid. An exemplary carrier fluid is oil, particularly, a fluorinated oil. In certain embodiments, the carrier fluid includes a surfactant, such as a fluorosurfactant. The droplets may be flowed through channels.

Any method known in the art may be used to attach the 5′ and the 3′ universal priming sites to the amplicons. In certain embodiments, the 5′ and 3′ ends of each of the plurality of amplicons include an adaptor sequence, in which the 5′ and the 3′ adaptor are different. Attaching the first oligonucleotide may involve providing the first oligonucleotide, the oligonucleotide including a first portion that is complementary to the adaptor attached at the 5′ end of the amplicon and a second portion that comprises the first universal primer site, and conducting an amplification reaction (e.g., PCR) between the amplicons of the first portion and the first oligonucleotides, to thereby produce amplicons comprising the first universal primer site attached to a 5′ end of the amplicons.

Attaching the second oligonucleotide may involve providing the second oligonucleotide, the oligonucleotide comprising a first portion that is complementary to the adaptor attached at the 3′ end of the amplicon and a second portion that comprises the second universal primer site, and conducting an amplification reaction (e.g., PCR) between the amplicons of the second portion and the second oligonucleotides, to thereby produce amplicons comprising the second universal primer site attached to a 3′ end of the amplicons.

In the case of droplets, each formed droplet includes either the first or second oligonucleotide and the reagents for an amplification reaction. Each droplet is caused to merge with a bolus of aqueous solution containing the amplicons, such that mixed droplets are formed that include amplicon and either the first or second oligonucleotide. In the case of droplets, post amplification, the droplets are pooled in a vessel and the amplicons are released from the droplets.

After pooling, the amplicons are sequenced. Sequencing may be by any method known in the art. Sequencing-by-synthesis is a common technique used in next generation procedures and works well with the instant invention. However, other sequencing methods can be used, including sequence-by-ligation, sequencing-by-hybridization; gel-based techniques and others. In general, sequencing involves hybridizing a primer to a template to form a template/primer duplex, contacting the duplex with a polymerase in the presence of a detectably-labeled nucleotides under conditions that permit the polymerase to add nucleotides to the primer in a template-dependent manner. Signal from the detectable label is then used as to identify the incorporated base and the steps are sequentially repeated in order to determine the linear order of nucleotides in the template. Exemplary detectable labels include radiolabels, florescent labels, enzymatic labels, etc. In particular embodiments, the detectable label may be an optically detectable label, such as a fluorescent label. Exemplary fluorescent labels include cyanine, rhodamine, fluorescien, coumarin, BODIPY, alexa, or conjugated multi-dyes.

### BRIEF DESCRIPTION OF THE DRAWINGS

FIGS. 1A-B shows an exemplary embodiment of a device for droplet formation.

FIGS. 2A-C shows an exemplary embodiment of merging two sample fluids according to methods of the invention.

FIGS. 3A-E show embodiments in which electrodes are used with methods of the invention to facilitate droplet merging. These figures show different positioning and different numbers of electrodes that may be used with methods of the invention. FIG. 3A shows a non-perpendicular orientation of the two channels at the merge site. FIGS. 3B-E shows a perpendicular orientation of the two channels at the merge site.

FIG. 4 shows an embodiment in which the electrodes are positioned beneath the channels. FIG. 4 also shows that an insulating layer may optionally be placed between the channels and the electrodes.

FIG. 5 shows an embodiment of forming a mixed droplet in the presence of electric charge and with use of a droplet track.

FIG. 6 shows a photograph capturing real-time formation of mixed droplets in the presence of electric charge and with use of a droplet track.

FIGS. 7A-B show an embodiment in which the second sample fluid includes multiple co-flowing streams of different fluids. FIG. 7A is with electrodes and FIG. 7B is without electrodes.

FIG. 8 shows a three channel embodiment for forming mixed droplets. This figure shows an embodiment without the presence of an electric field.

FIG. 9 shows a three channel embodiment for forming mixed droplets. FIG. 9 shows an embodiment that employs an electric field to facilitate droplet merging.

FIG. 10 shows a three channel embodiment for forming mixed droplets. This figure shows a droplet not merging with a bolus of the second sample fluid. Rather, the bolus of the second sample fluid enters the channel as a droplet and merges with a droplet of the first sample fluid at a point past the intersection of the channels.

FIGS. 11A-C show embodiments in which the size of the orifice at the merge point for the channel through which the second sample fluid flows may be the smaller, the same size as, or larger than the cross-sectional dimension of the channel through which the immiscible carrier fluid flows.

FIGS. 12A-B are a set of photographs showing an arrangement that was employed to form a mixed droplet in which a droplet of a first fluid was brought into contact with a bolus of a second sample fluid stream, in which the bolus was segmented from the second fluid stream and merged with the droplet to form a mixed droplet in an immiscible carrier fluid. FIG. 12A shows the droplet approaching the growing bolus of the second fluid stream. FIG. 12B shows the droplet merging and mixing with the bolus of the second fluid stream.

FIGS. 13A-B show a droplet track that was employed with methods of the invention to steer droplets away from the center streamlines and toward the emerging bolus of the second fluid on entering the merge area. These figures show that a mixed droplet was formed without the presence of electric charge and with use of a droplet track.

### DETAILED DESCRIPTION

The invention generally relates to methods for sequencing a nucleic acid template from both a 5′ and a 3′ end in a single sequencing reaction. In certain embodiments, methods of the invention involve amplifying a nucleic acid template to produce a plurality of amplicons, splitting the amplicons into first and second portions, attaching a first oligonucleotide including a first universal primer site to a 5′ end of the amplicons in the first portion, and attaching a second oligonucleotide including a second universal primer site to a 3′ end of the amplicons in the second portion. The first and second primer sites may be the same or they may be different. The method additionally involves pooling the first and second portions, and sequencing the pooled amplicons, thereby sequencing a nucleic acid template from both a 5′ and a 3′ end in a single sequencing reaction.

### Nucleic Acids

DNA generally is acquired from a sample or a subject. Target molecules for labeling and/or detection according to the methods of the invention include, but are not limited to, genetic and proteomic material, such as DNA, genomic DNA, RNA, expressed RNA and/or chromosome(s). Methods of the invention are applicable to DNA from whole cells or to portions of genetic or proteomic material obtained from one or more cells. For a subject, the sample may be obtained in any clinically acceptable manner, and the nucleic acid templates are extracted from the sample by methods known in the art. Nucleic acid templates can be obtained as described in U.S. Patent Application Publication Number US2002/0190663 A1, published Oct. 9, 2003. Generally, nucleic acid can be extracted from a biological sample by a variety of techniques such as those described by Maniatis, et al. (Molecular Cloning: A Laboratory Manual, Cold Spring Harbor, N.Y., pp. 280-281, 1982), the contents of which are incorporated by reference herein in their entirety.

Nucleic acid templates include deoxyribonucleic acid (DNA) and/or ribonucleic acid (RNA). Nucleic acid templates can be synthetic or derived from naturally occurring sources. In one embodiment, nucleic acid templates are isolated from a biological sample containing a variety of other components, such as proteins, lipids and non-template nucleic acids. Nucleic acid templates can be obtained from any cellular material, obtained from an animal, plant, bacterium, fungus, or any other cellular organism. Biological samples for use in the present invention include viral particles or preparations. Nucleic acid templates can be obtained directly from an organism or from a biological sample obtained from an organism, e.g., from blood, urine, cerebrospinal fluid, seminal fluid, saliva, sputum, stool and tissue. In a particular embodiment, nucleic acid is obtained from fresh frozen plasma (FFP). Any tissue or body fluid specimen may be used as a source for nucleic acid for use in the invention. Nucleic acid templates can also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA.

Generally, nucleic acid obtained from biological samples is fragmented to produce suitable fragments for analysis. An advantage of methods of the invention is that they can be performed on nucleic acids that have not been fragmented.

However, in certain embodiments, nucleic acids are fragmented prior to performing methods of the invention. In one embodiment, nucleic acid from a biological sample is fragmented by sonication. Generally, individual nucleic acid template molecules can be from about 5 bases to about 20 kb.

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is non-denaturing, can act to solubilize the sample. Detergents may be ionic or nonionic. Examples of nonionic detergents include triton, such as the Triton® X series (Triton® X-100 t-Oct-C6H4-(OCH₂—CH₂)xOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycolsorbitan monooleate, polidocanol, ndodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant.

Lysis or homogenization solutions may further contain other agents, such as reducing agents. Examples of such reducing agents include dithiothreitol (DTT), .beta.-mercaptoethanol, DTE, GSH, cysteine, cysteamine, tricarboxyethyl phosphine (TCEP), or salts of sulfurous acid. Once obtained, the nucleic acid is denatured by any method known in the art to produce single stranded nucleic acid templates and a pair of first and second oligonucleotides is hybridized to the single stranded nucleic acid template such that the first and second oligonucleotides flank a target region on the template.

### Generating Amplicons

Methods of the invention involve amplifying a nucleic acid template to produce a plurality of amplicons. In certain embodiments, the 5′ and 3′ ends of each of the plurality of amplicons include an adaptor sequence, in which the 5′ and the 3′ adaptor are different. Any adaptor sequences may be used so long as the adaptors on the 5′ end are different from the adaptors on the 3′ end. Generally, the adaptors will be poly(A) and poly (T) tails. Other more unique adaptor sequences may be used. All of the 5′ adaptors are the same and all of the 3′ adaptors are the same.

The adaptors may be attached using any method known in the art. In certain embodiments, the adaptor sequences are attached to the template with an enzyme. The enzyme may be a ligase or a polymerase. The ligase may be any enzyme capable of ligating an oligonucleotide (RNA or DNA) to the template nucleic acid molecule. Suitable ligases include T4 DNA ligase and T4 RNA ligase (such ligases are available commercially, from New England Biolabs). Methods for using ligases are well known in the art. The polymerase may be any enzyme capable of adding nucleotides to the 3′ and the 5′ terminus of template nucleic acid molecules.

The ligation may be blunt ended. The end of the copy may be treated with a polymerase and dATP to form a template independent addition to the 3′-end of the copy, thus producing a single A overhanging. This single A is used to guide ligation of fragments with a single T overhanging from the 5′-end in a method referred to as T-A cloning.

Once adaptor have been ligated to the template, the template is amplified to produce a plurality of amplicons. Amplification refers to production of additional copies of a nucleic acid sequence and is generally carried out using polymerase chain reaction or other technologies well known in the art (e.g., Dieffenbach and Dveksler, PCR Primer, a Laboratory Manual, Cold Spring Harbor Press, Plainview, N.Y. [1995]). The amplification reaction may be any amplification reaction known in the art that amplifies nucleic acid molecules, such as polymerase chain reaction, nested polymerase chain reaction, polymerase chain reaction-single strand conformation polymorphism, ligase chain reaction (Barany F. (1991) PNAS 88:189-193; Barany F. (1991) PCR Methods and Applications 1:5-16), ligase detection reaction (Barany F. (1991) PNAS 88:189-193), strand displacement amplification and restriction fragments length polymorphism, transcription based amplification system, nucleic acid sequence-based amplification, rolling circle amplification, and hyper-branched rolling circle amplification. Polymerase chain reaction (PCR) refers to methods by K. B. Mullis (U.S. Pat. Nos. 4,683,195 and 4,683,202, hereby incorporated by reference) for increasing concentration of a segment of a target sequence in a mixture of genomic DNA without cloning or purification. The process for amplifying the target sequence includes introducing an excess of primers (oligonucleotides) to a DNA mixture containing a desired target sequence, followed by a precise sequence of thermal cycling. The present invention includes, but is not limited to, various PCR strategies as are known in the art, for example QPCR, multiplex PCR, asymmetric PCR, nested PCR, hotstart PCR, touchdown PCR, assembly PCR, digital PCR, allele specific PCR, methylation specific PCR, reverse transcription PCR, helicase dependent PCR, inverse PCR, intersequence specific PCR, ligation mediated PCR, mini primer PCR, and solid phase PCR, emulsion PCR, and PCR as performed in a thermocycler, droplets, microfluidic reaction chambers, flow cells and other microfluidic devices.

Splitting the Amplicons into First and Second Portions

Methods of the invention involve splitting the amplicons into first and second portions. Any technique known in the art may be used to split the amplicons into first and second portions. Exemplary compartmentalizing techniques are shown for example in, Griffiths et al. (U.S. Pat. No. 7,968,287) and Link et al. (U.S. patent application number 2008/0014589), the content of each of which is incorporated by reference herein in its entirety.

In certain embodiments, splitting involves compartmentalizing the amplicons into compartmentalized portions. In particular embodiments, the compartmentalized portions are droplets and compartmentalizing involves introducing the aqueous solution of amplicons to a stream of droplets. Each droplet includes either the first or second oligonucleotide and the reagents for an amplification reaction. The first oligonucleotide includes a first portion that is complementary to the adaptor at the 5′ end of the amplicon and a second portion that includes a universal primer site. The second oligonucleotide includes a first portion that is complementary to the adaptor at the 3′ end of the amplicon and a universal primer site. The universal primer site of the first oligonucleotide and the universal primer site of the second oligonucleotide are different. Each droplet is caused to merge with a bolus of aqueous solution containing the amplicons, such that mixed droplets are formed that include amplicon and either the first or second oligonucleotide.

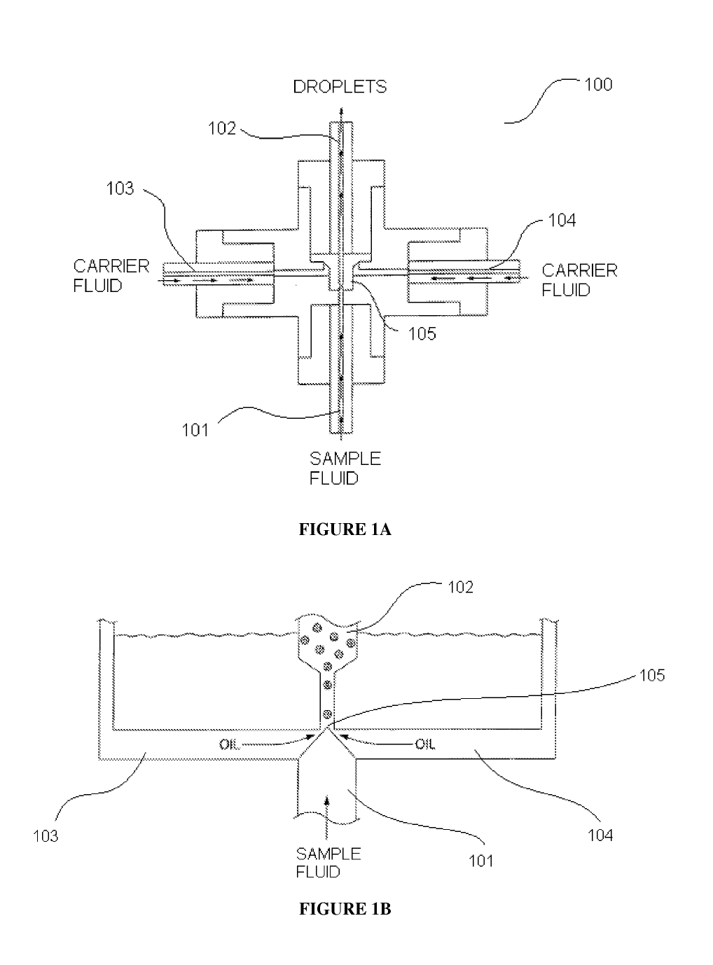

Sample droplets may be formed by any method known in the art. The droplets are aqueous droplets that are surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803), Anderson et al. (U.S. Pat. No. 7,041,481 and which reissued as RE41,780) and European publication number EP2047910 to Raindance Technologies Inc. The content of each of which is incorporated by reference herein in its entirety. FIGS. 1A-B show an exemplary embodiment of a device 100 for droplet formation. Device 100 includes an inlet channel 101, and outlet channel 102, and two carrier fluid channels 103 and 104. Channels 101, 102, 103, and 104 meet at a junction 105. Inlet channel 101 flows sample fluid to the junction 105. Carrier fluid channels 103 and 104 flow a carrier fluid that is immiscible with the sample fluid to the junction 105. Inlet channel 101 narrows at its distal portion wherein it connects to junction 105 (See FIG. 1B). Inlet channel 101 is oriented to be perpendicular to carrier fluid channels 103 and 104. Droplets are formed as sample fluid flows from inlet channel 101 to junction 105, where the sample fluid interacts with flowing carrier fluid provided to the junction 105by carrier fluid channels 103 and 104. Outlet channel 102 receives the droplets of sample fluid surrounded by carrier fluid.

The sample fluid is typically an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. Any liquid or buffer that is physiologically compatible with nucleic acid molecules can be used. The carrier fluid is one that is immiscible with the sample fluid. The carrier fluid can be a non-polar solvent, decane (e.g., tetradecane or hexadecane), fluorocarbon oil, silicone oil or another oil (for example, mineral oil).

In certain embodiments, the carrier fluid contain some or more additives, such as agents which reduce surface tensions (surfactants). Surfactants can include Tween, Span, fluorosurfactants, and other agents that are soluble in oil relative to water. In some applications, performance is improved by adding a second surfactant to the sample fluid. Surfactants can aid in controlling or optimizing droplet size, flow and uniformity, for example by reducing the shear force needed to extrude or inject droplets into an intersecting channel. This can affect droplet volume and periodicity, or the rate or frequency at which droplets break off into an intersecting channel. Furthermore, the surfactant can serve to stabilize aqueous emulsions in fluorinated oils from coalescing.

In certain embodiments, the droplets may be coated with a surfactant. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglyceryl esters of natural fatty acids, propylene glycol, sorbitol, polyoxyethylenated sorbitol esters, polyoxyethyleneglycol esters, etc.) and alkanolamines (e.g., diethanolamine-fatty acid condensates and isopropanolamine-fatty acid condensates). In certain embodiments, the carrier fluid may be caused to flow through the outlet channel so that the surfactant in the carrier fluid coats the channel walls. In one embodiment, the fluorosurfactant can be prepared by reacting the perfluorinated polyether DuPont Krytox 157 FSL, FSM, or FSH with aqueous ammonium hydroxide in a volatile fluorinated solvent. The solvent and residual water and ammonia can be removed with a rotary evaporator. The surfactant can then be dissolved (e.g., 2.5 wt %) in a fluorinated oil (e.g., Flourinert (3M)), which then serves as the carrier fluid.

After formation of the droplets containing either the first or second oligonucleotide, the droplets are contacted with a flow of a second sample fluid stream including the amplicons. Contact between the droplets and the fluid stream results in a portion of the fluid stream integrating with the droplets to form a mixed droplet. Each mixed droplet includes either the first or second oligonucleotide and a plurality of amplicons.

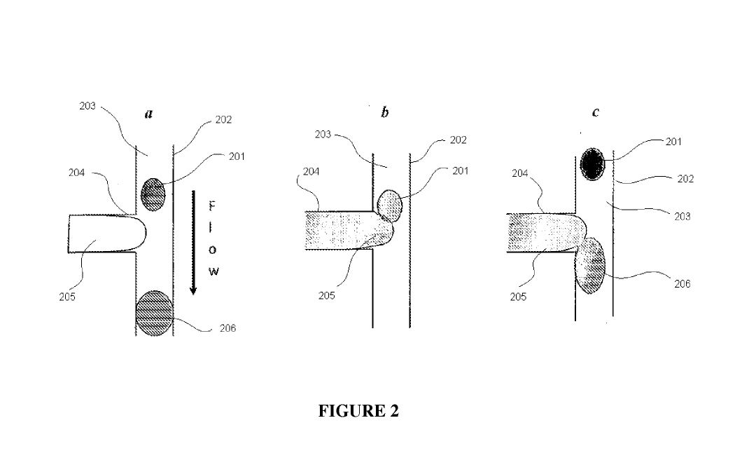

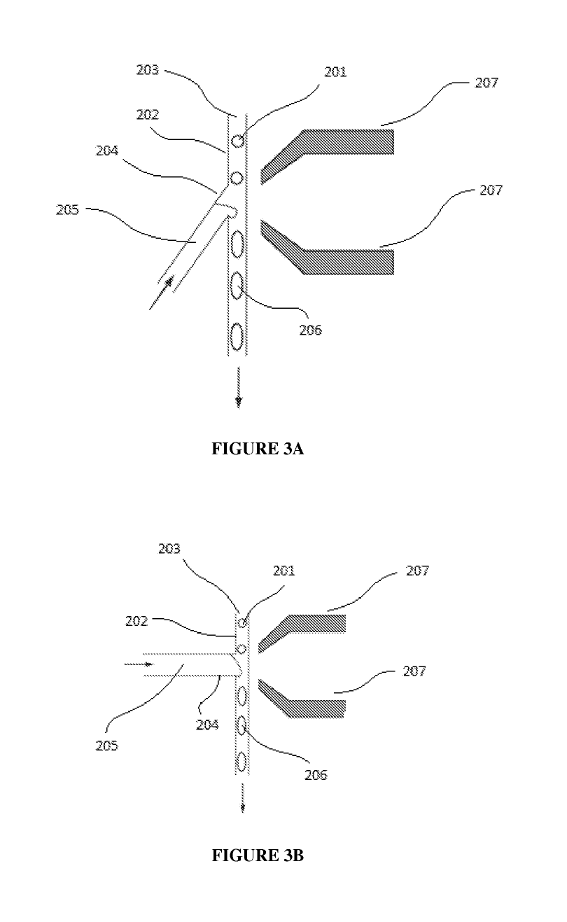

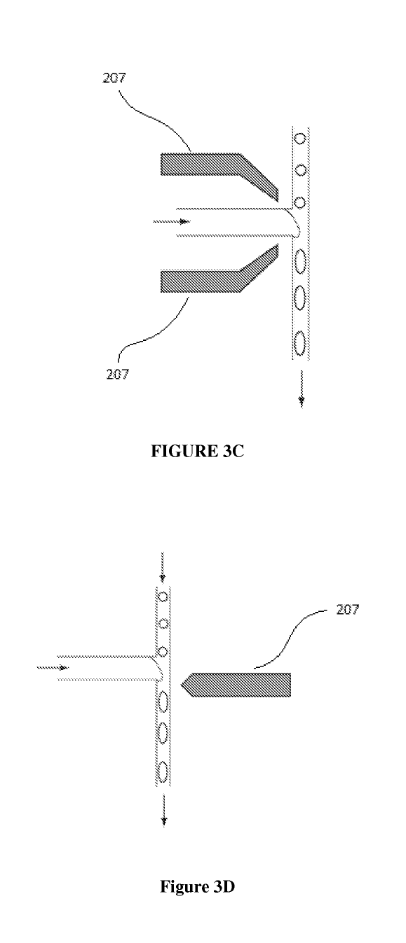

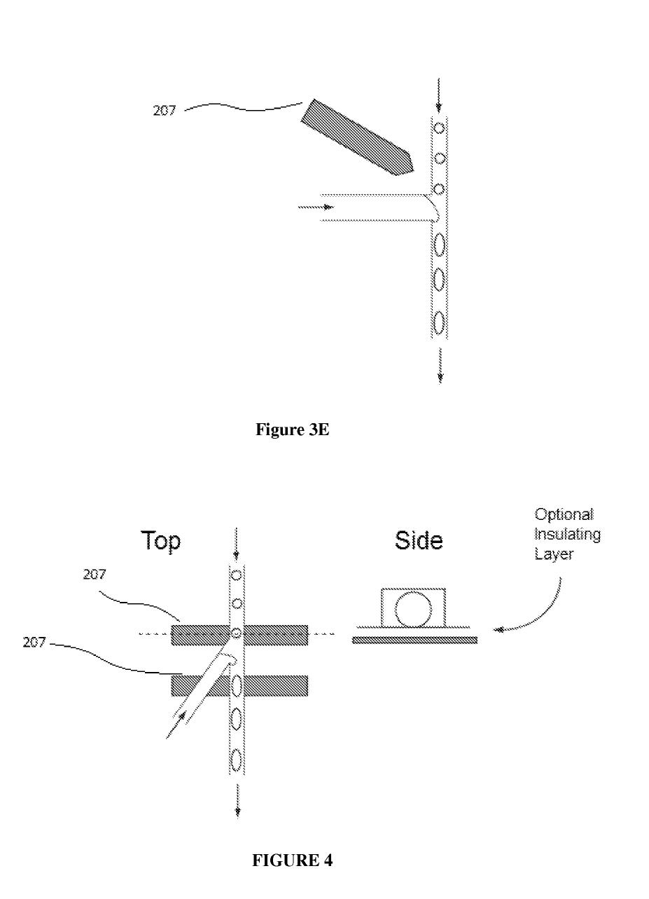

FIG. 2 provides a schematic showing merging of sample fluids according to methods of the invention. Droplets 201 including either the first or second oligonucleotides flow through a first channel 202 separated from each other by immiscible carrier fluid and suspended in the immiscible carrier fluid 203. The droplets 201 are delivered to the merge area, i.e., junction of the first channel 202 with the second channel 204, by a pressure-driven flow generated by a positive displacement pump. While droplet 201 arrives at the merge area, a bolus of a second sample fluid 205 is protruding from an opening of the second channel 204 into the first channel 202 (FIG. 2A). FIGS. 2 and 3B show the intersection of channels 202 and 204 as being perpendicular. However, any angle that results in an intersection of the channels 202 and 204 may be used, and methods of the invention are not limited to the orientation of the channels 202 and 204 shown in FIG. 2. For example, FIG. 3A shows an embodiment in which channels 202 and 204 are not perpendicular to each other. The droplets 201 shown in FIG. 2 are monodispersive, but non-monodispersive drops are useful in the context of the invention as well.

The bolus of the second sample fluid stream 205 continues to increase in size due to pumping action of a positive displacement pump connected to channel 204, which outputs a steady stream of the second sample fluid 205 into the merge area. The flowing droplet 201 containing the first sample fluid eventually contacts the bolus of the second sample fluid 205 that is protruding into the first channel 202. Contact between the two sample fluids results in a portion of the second sample fluid 205 being segmented from the second sample fluid stream and joining with the first sample fluid droplet 201 to form a mixed droplet 206 (FIGS. 2B-C). FIG. 12 shows an arrangement that was employed to form a mixed droplet in which a droplet of a first fluid was brought into contact with a bolus of a second sample fluid stream, in which the bolus was segmented from the second fluid stream and merged with the droplet to form a mixed droplet in an immiscible carrier fluid. FIG. 12A shows the droplet approaching the growing bolus of the second fluid stream. FIG. 12B shows the droplet merging and mixing with the bolus of the second fluid stream. In certain embodiments, each incoming droplet 201 of first sample fluid is merged with the same amount of second sample fluid 205. In order to achieve the merge of the first and second sample fluids, the interface separating the fluids must be ruptured. In certain embodiments, this rupture can be achieved through the application of an electric charge. In certain embodiments, the rupture will result from application of an electric field. In certain embodiments, the rupture will be achieved through non-electrical means, e.g. by hydrophobic/hydrophilic patterning of the surface contacting the fluids.

In certain embodiments, an electric charge is applied to the first and second sample fluids (FIGS. 3A-E). Any number of electrodes may be used with methods of the invention in order to apply an electric charge. FIGS. 3A-C show embodiments that use two electrodes 207. FIGS. 3D-E show embodiments that use one electrode 207. The electrodes 207 may positioned in any manner and any orientation as long as they are in proximity to the merge region. In FIGS. 3A-B and D, the electrodes 207 are positioned across from the merge junction. In FIGS. 3C and E, the electrodes 207 are positioned on the same side as the merge junction. In certain embodiments, the electrodes are located below the channels (FIG. 4). In certain embodiments, the electrodes are optionally separated from the channels by an insulating layer (FIG. 4).

Description of applying electric charge to sample fluids is provided in Link et al. (U.S. patent application number 2007/0003442) and European Patent Number EP2004316 to Raindance Technologies Inc, the content of each of which is incorporated by reference herein in its entirety. Electric charge may be created in the first and second sample fluids within the carrier fluid using any suitable technique, for example, by placing the first and second sample fluids within an electric field (which may be AC, DC, etc.), and/or causing a reaction to occur that causes the first and second sample fluids to have an electric charge, for example, a chemical reaction, an ionic reaction, a photocatalyzed reaction, etc.

The electric field, in some embodiments, is generated from an electric field generator, i.e., a device or system able to create an electric field that can be applied to the fluid. The electric field generator may produce an AC field (i.e., one that varies periodically with respect to time, for example, sinusoidally, saw tooth, square, etc.), a DC field (i.e., one that is constant with respect to time), a pulsed field, etc. The electric field generator may be constructed and arranged to create an electric field within a fluid contained within a channel or a microfluidic channel. The electric field generator may be integral to or separate from the fluidic system containing the channel or microfluidic channel, according to some embodiments.

Techniques for producing a suitable electric field (which may be AC, DC, etc.) are known to those of ordinary skill in the art. For example, in one embodiment, an electric field is produced by applying voltage across a pair of electrodes, which may be positioned on or embedded within the fluidic system (for example, within a substrate defining the channel or microfluidic channel), and/or positioned proximate the fluid such that at least a portion of the electric field interacts with the fluid. The electrodes can be fashioned from any suitable electrode material or materials known to those of ordinary skill in the art, including, but not limited to, silver, gold, copper, carbon, platinum, tungsten, tin, cadmium, nickel, indium tin oxide (“ITO”), etc., as well as combinations thereof. In some cases, transparent or substantially transparent electrodes can be used.

The electric field facilitates rupture of the interface separating the second sample fluid 205 and the droplet 201. Rupturing the interface facilitates merging of the bolus of the second sample fluid 205 and the first sample fluid droplet 201 (FIG. 2B). The forming mixed droplet 206 continues to increase in size until it a portion of the second sample fluid 205 breaks free or segments from the second sample fluid stream prior to arrival and merging of the next droplet containing the first sample fluid (FIG. 2C). The segmenting of the portion of the second sample fluid from the second sample fluid stream occurs as soon as the force due to the shear and/or elongational flow that is exerted on the forming mixed droplet 206 by the immiscible carrier fluid overcomes the surface tension whose action is to keep the segmenting portion of the second sample fluid connected with the second sample fluid stream. The now fully formed mixed droplet 206 continues to flow through the first channel 206.

FIG. 5 illustrates an embodiment in which a drop track 208 is used in conjunction with electrodes 207 to facilitate merging of a portion of the second fluid 205 with the droplet 201. Under many circumstances it is advantageous for microfluidic channels to have a high aspect ratio defined as the channel width divided by the height. One advantage is that such channels tend to be more resistant against clogging because the “frisbee” shaped debris that would otherwise be required to occlude a wide and shallow channel is a rare occurrence. However, in certain instances, high aspect ratio channels are less preferred because under certain conditions the bolus of liquid 205 emerging from the continuous phase channel into merge may dribble down the side of the merge rather than snapping off into clean uniform merged droplets 206. An aspect of the invention that ensures that methods of the invention function optimally with high aspect ratio channels is the addition of droplets “tracks” 208 that both guide the droplets toward the emerging bolus 205 within the merger and simultaneously provides a microenvironment more suitable for the snapping mode of droplet generation. A droplet track 208 is a trench in the floor or ceiling of a conventional rectangular microfluidic channel that can be used either to improve the precision of steering droplets within a microfluidic channel and also to steer droplets in directions normally inaccessible by flow alone. The track could also be included in a side wall. FIG. 5 shows a cross-section of a channel with a droplet track 208. The channel height (marked “h”) is the distance from the channel floor to the ceiling/bottom of the track 208, and the track height is the distance from the bottom of the track to the channel floor ceiling (marked “t”). Thus the total height within the track is the channel height plus the track height. In a preferred embodiment, the channel height is substantially smaller than the diameter of the droplets contained within the channel, forcing the droplets into a higher energy “squashed” conformation. Such droplets that encounter a droplet track 208 will expand into the track spontaneously, adopting a lower energy conformation with a lower surface area to volume ratio. Once inside a track, extra energy is required to displace the droplet from the track back into the shallower channel. Thus droplets will tend to remain inside tracks along the floor and ceiling of microfluidic channels even as they are dragged along with the carrier fluid in flow. If the direction along the droplet track 208 is not parallel to the direction of flow, then the droplet experiences both a drag force in the direction of flow as well as a component perpendicular to the flow due to surface energy of the droplet within the track. Thus the droplet within a track can displace at an angle relative to the direction of flow which would otherwise be difficult in a conventional rectangular channel.

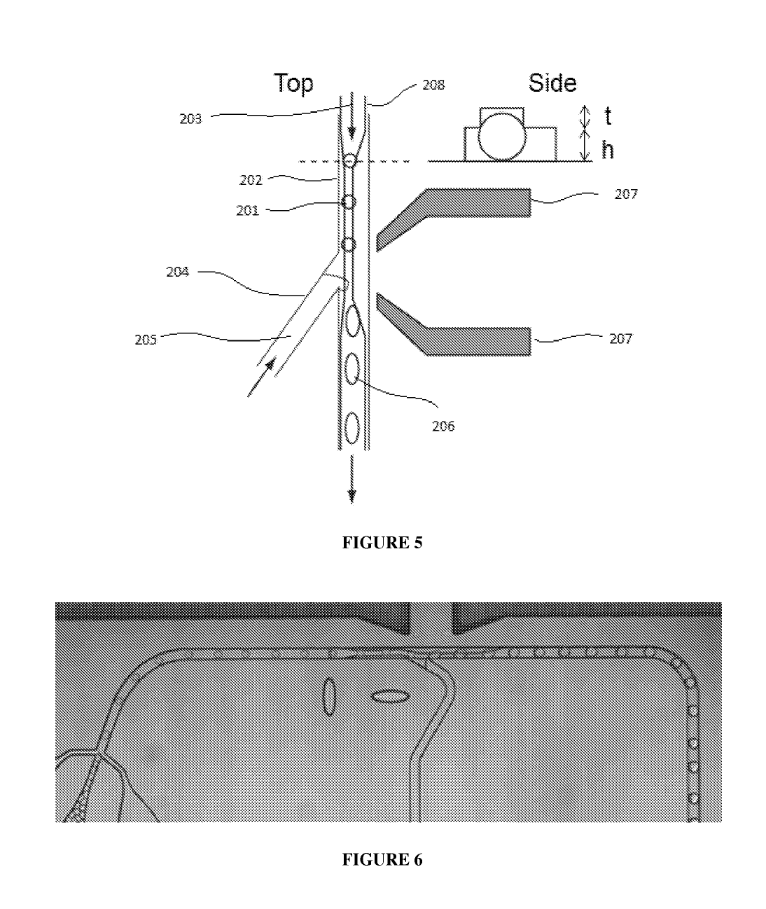

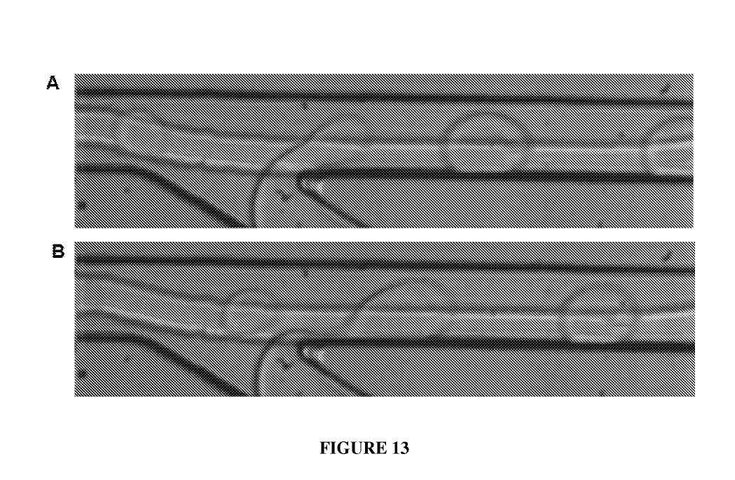

In FIG. 5, droplets 201 of the first sample fluid flow through a first channel 202 separated from each other by immiscible carrier fluid and suspended in the immiscible carrier fluid 203. The droplets 201 enter the droplet track 208 which steers or guides the droplets 201 close to the where the bolus of the second fluid 205 is emerging from the second channel 204. The steered droplets 201 in the droplet track 208 are delivered to the merge area, i.e., junction of the first channel 202 with the second channel 204, by a pressure-driven flow generated by a positive displacement pump. While droplet 201 arrives at the merge area, a bolus of a second sample fluid 205 is protruding from an opening of the second channel 204 into the first channel 202. The bolus of the second sample fluid stream 205 continues to increase in size due to pumping action of a positive displacement pump connected to channel 204, which outputs a steady stream of the second sample fluid 205 into the merge area. The flowing droplet 201 containing the first sample fluid eventually contacts the bolus of the second sample fluid 205 that is protruding into the first channel 202. The contacting happens in the presence of electrodes 207, which provide an electric charge to the merge area, which facilitates the rupturing of the interface separating the fluids. Contact between the two sample fluids in the presence of the electric change results in a portion of the second sample fluid 205 being segmented from the second sample fluid stream and joining with the first sample fluid droplet 201 to form a mixed droplet 206. The now fully formed mixed droplet 206 continues to flow through the droplet trap 208 and through the first channel 203. FIG. 6 shows a droplet track that was employed with methods of the invention to steer droplets away from the center streamlines and toward the emerging bolus of the second fluid on entering the merge area. This figure shows that a mixed droplet was formed in the presence of electric charge and with use of a droplet track. FIGS. 13A-B show a droplet track that was employed with methods of the invention to steer droplets away from the center streamlines and toward the emerging bolus of the second fluid on entering the merge area. These figures show that a mixed droplet was formed without the presence of electric charge and with use of a droplet track.

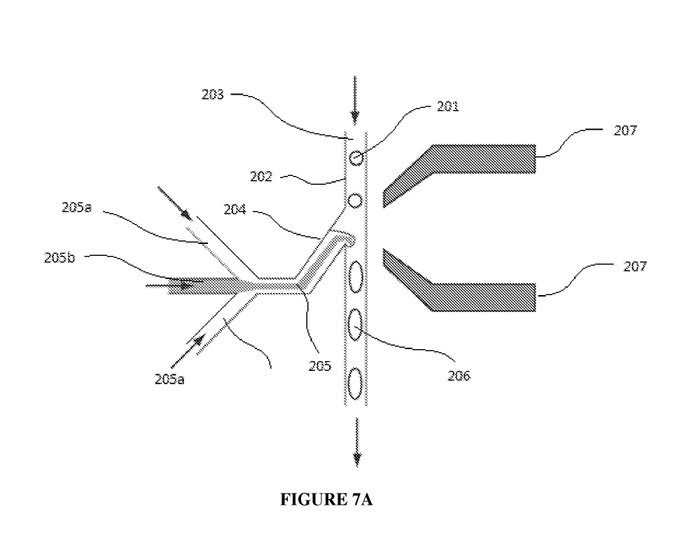

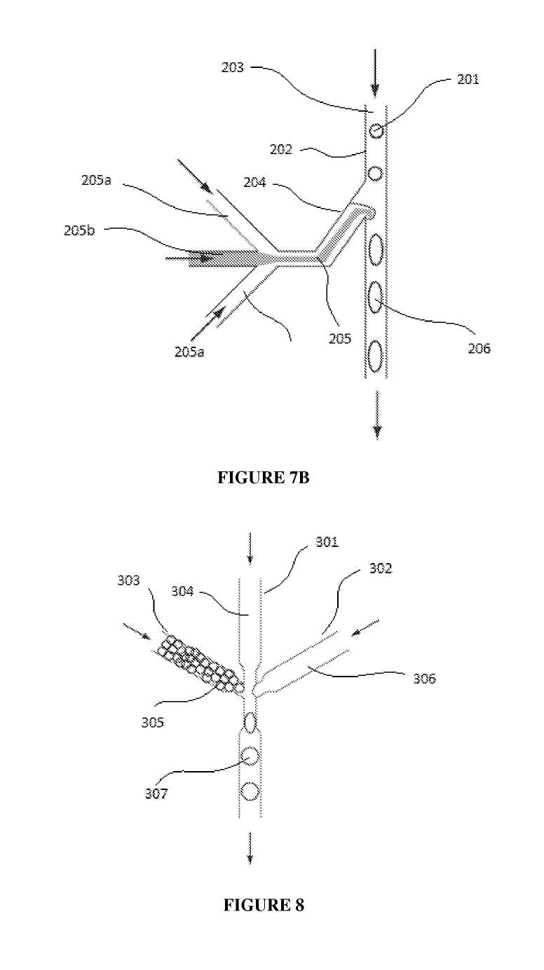

In certain embodiments, the second sample fluid 205 may consist of multiple co-flowing streams of different fluids. Such embodiments are shown in FIGS. 7A-B. FIG. 7A is with electrodes and FIG. 7B is without electrodes. In this embodiments, sample fluid 205 is a mixture of two different sample fluids 205a and 205b. Samples fluids 205a and 205b mix upstream in channel 204 and are delivered to the merge area as a mixture. A bolus of the mixture then contacts droplet 201. Contact between the mixture in the presence or absence of the electric change results in a portion of the mixed second sample fluid 205 being segmented from the mixed second sample fluid stream and joining with the first sample fluid droplet 201 to form a mixed droplet 206. The now fully formed mixed droplet 206 continues to flow through the through the first channel 203.

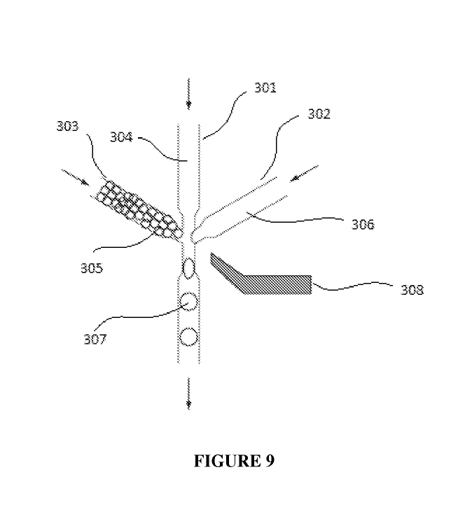

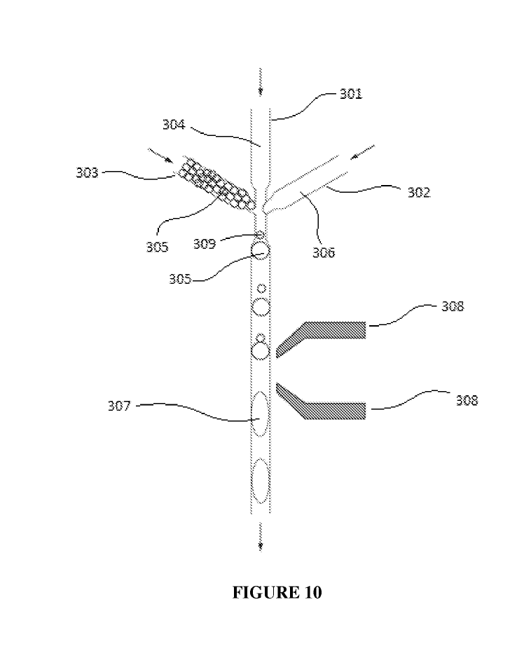

FIG. 8 shows a three channel embodiment. In this embodiment, channel 301 is flowing immiscible carrier fluid 304. Channels 302 and 303 intersect channel 301. FIG. 8 shows the intersection of channels 301-303 as not being perpendicular, and angle that results in an intersection of the channels 301-303 may be used. In other embodiments, the intersection of channels 301-303 is perpendicular. Channel 302 include a plurality of droplets 305 of a first sample fluid, while channel 303 includes a second sample fluid stream 306. In certain embodiments, a droplet 305 is brought into contact with a bolus of the second sample fluid 306 in channel 301 under conditions that allow the bolus of the second sample fluid 306 to merge with the droplet 305 to form a mixed droplet 307 in channel 301 that is surrounded by carrier fluid 304. In certain embodiments, the merging is in the presence of an electric charge provided by electrode 308 (FIG. 9). In certain embodiments, channel 301 narrows in the regions in proximity to the intersection of channels 301-303. However, such narrowing is not required and the described embodiments can be performed without a narrowing of channel 301. In certain embodiments, it is desirable to cause the droplet 305 and the bolus of the second sample fluid 306 to enter channel 301 without merging, as shown in FIG. 10. In these embodiments, the bolus of the second sample fluid 306 breaks-off from the second sample fluid stream and forms a droplet 309. Droplet 309 travels in the carrier fluid 304 with droplet 305 that has been introduced to channel 301 from channel 303 until conditions in the channel 301 are adjusted such that droplet 309 is caused to merge with droplet 305. Such a change in conditions can be turbulent flow, change in hydrophobicity, or as shown in FIG. 10, application of an electric charge from an electrode 308 to the fluids in channel 301. Application of the electric charge, causes droplets 309 and 305 to merge and form mixed droplet 307.

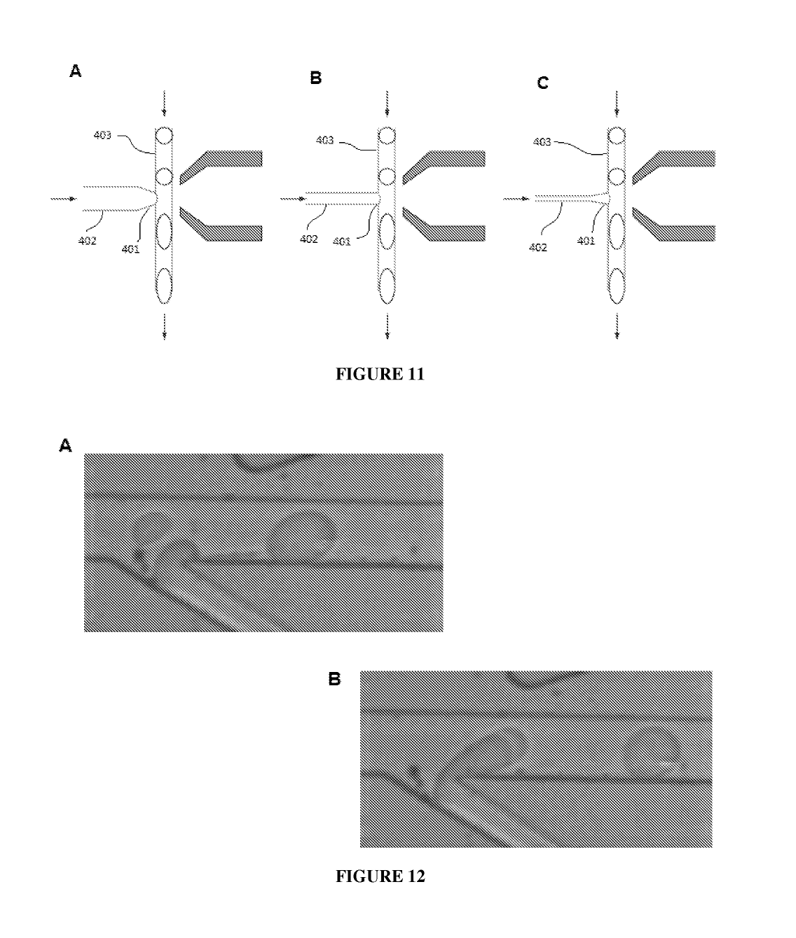

In embodiments of the invention, the size of the orifice at the merge point for the channel through which the second sample fluid flows may be the smaller, the same size as, or larger than the cross-sectional dimension of the channel through which the immiscible carrier fluid flows. FIGS. 11A-C illustrate these embodiments. FIG. 11A shows an embodiment in which the orifice 401 at the merge point for the channel 402 through which the second sample fluid flows is smaller than the cross-sectional dimension of the channel 403 through which the immiscible carrier fluid flows. In these embodiments, the orifices 401 may have areas that are 90% or less than the average cross-sectional dimension of the channel 403. FIG. 11B shows an embodiment in which the orifice 401 at the merge point for the channel 402 through which the second sample fluid flows is the same size as than the cross-sectional dimension of the channel 403 through which the immiscible carrier fluid flows. FIG. 11C shows an embodiment in which the orifice 401 at the merge point for the channel 402 through which the second sample fluid flows is larger than the cross-sectional dimension of the channel 403 through which the immiscible carrier fluid flows.

### Attaching the 5′ Universal Primer Site and the 3′ Universal Primer Site

Methods of the invention further involve amplifying the target genetic material in each droplet using appropriate primers and amplification strategies to attach the universal sequencing primer and accomplish bidirectional sequencing. Generally, to effect amplification, primers are annealed to their complementary sequence within the target molecule. Following annealing, the primers are extended with a polymerase so as to form a new pair of complementary strands. The steps of denaturation, primer annealing and polymerase extension can be repeated many times (i.e., denaturation, annealing and extension constitute one cycle; there can be numerous cycles) to obtain a high concentration of an amplified segment of a desired target sequence. The length of the amplified segment of the desired target sequence is determined by relative positions of the primers with respect to each other and by cycling parameters, and therefore, this length is a controllable parameter.

Primers can be prepared by a variety of methods including but not limited to cloning of appropriate sequences and direct chemical synthesis using methods well known in the art (Narang et al., Methods Enzymol., 68:90 (1979); Brown et al., Methods Enzymol., 68:109 (1979)). Primers can also be obtained from commercial sources such as Operon Technologies, Amersham Pharmacia Biotech, Sigma, and Life Technologies. The primers can have an identical melting temperature. The lengths of the primers can be extended or shortened at the 5′ end or the 3′ end to produce primers with desired melting temperatures. Also, the annealing position of each primer pair can be designed such that the sequence and, length of the primer pairs yield the desired melting temperature. The simplest equation for determining the melting temperature of primers smaller than 25 base pairs is the Wallace Rule (Td=2(A+T)+4(G+C)). Another method for determining the melting temperature of primers is the nearest neighbor method (̂ a b c John Santa Lucia Jr. (1998). “A unified view of polymer, dumbbell, and oligonucleotide DNA nearest neighbor thermodynamics”. Proc. Natl. Acad. Sci. USA 95 (4): 1460-5. doi:10.1073/pnas.95.4.1460. PMID 9465037). Computer programs can also be used to design primers, including but not limited to Array Designer Software (Arrayit Inc.), Oligonucleotide Probe Sequence Design Software for Genetic Analysis (Olympus Optical Co.), Net Primer, and DNAs is from Hitachi Software Engineering. The TM (melting or annealing temperature) of each primer is calculated using software programs such as Oligo Design, available from Invitrogen Corp.

In one embodiment, a universal primer site is attached to the target DNA sequence amplicon using PCR through at two stages. A first stage is for the attachment of an adapter sequence, and a second stage is for the attachment of the universal primer sequence. Adapter primer sequences are synthesized having a 3′ portion designed to hybridize to the target DNA molecules, and a 5′ end having a complimentary sequence to a portion of a universal sequencing oligonucleotides. Universal sequencing oligonucleotides are synthesized wherein the 3′ portion is complimentary to the 5′ end of the adaptor region, and the 5′ portion comprises the universal sequencing primer sequence. Target DNA sequences are amplified using, for example, primers with adaptor sequences: 3′-CGCTCTTCCGATCTCTG (SEQ ID NO: 1)-AMPLICON-CAGTCTAGCCTTCTCGT (SEQ ID NO: 2)-5′ in which the tail ends shown in bold comprise the adaptor sequences on all of the amplicons generated from template DNA strands. In one embodiment, the 3′ adapter can be the same as the 5′ adapter. In another embodiment, the 3′ adapter sequence can be different from the 5′ adapter sequence. The sequence portions hybridizing to the target DNA are optimized for hybridizing to the sequences of the amplicon. After the first amplification stage, amplicons will have a 5′ end and a 3′ end with the same, or different, adapter sequences.

Purification of the resulting amplicons is accomplished by methods well known in the art, for example using PCR product purification kits (Qiagen). The purified PCR product is portioned into two samples using, for example, automated means, for example microfluidic devices described herein, wherein the amplicons are compartmentalized into droplets and the population of droplets is portioned into a first population and a second population.

In the second PCR amplification stage, the first population amplicons are PCR amplified with a first set of universal sequencing primers. These primers anneal at the 5′ end of the target amplicon at the position of the 5′ adapter sequence for the primer annealing step of the second PCR amplification. The sequence below, for example, comprises a portion complimentary to the sequence of the 5′ end of the 5′ adaptor (in bold) in the amplicon: 5′-CCATCTCATCCCTGCGTGTCTCCGACTCAGCGCTCTTCCGATCTCTG-3′ (SEQ ID NO: 3). The sequence below comprises a portion complimentary to the sequence of the 5′ end of the 3′ adaptor (in bold) in the amplicon: 3′-CAGTCTAGCCTTCTCGTGACTCTGACGGTTCCGTGTGTCCCCTATCC-5′ (SEQ ID NO: 4). The second population amplicons are PCR amplified with a second set of universal sequencing oligonucleotide primers. These primers anneal at the 3′ end (complimentary strand) of target the amplicon at the position of the 3′ adapter sequence for the primer annealing step of the second amplification. The sequence below comprises a portion complimentary to the sequence of the 5′ end of the 5′ adaptor (in bold) in the amplicon: 5′-CCTATCCCCTGTGTGCCTTGGCAGTCTCAGCGCTCTTCCGATCTCTG-3′ (SEQ ID NO: 5). The sequence below comprises a portion complimentary to the sequence of the 5′ end of the 3′ adaptor (in bold) in the amplicon: 3′-CAGTCTAGCCTTCTCGTGACTCAGCCTCTGTGCGTCCCTACTCTACC-5′ (SEQ ID NO: 7). The resulting populations of amplicons have a universal sequencing primer attached to the 5′ end of the amplicon originating from the target DNA, and a second universal sequencing primer attached to the 5′ end of the amplicon originating from target DNA. The universal sequencing primers are attached, allowing for sequencing proceeds in both strands in a 5′ to 3′ direction. Preferably the sequence of the 5′ universal sequencing primer is different than the 3′ universal sequencing primer to facilitate strand differentiation in the sequencing stage. Any adapter sequence can be designed that is appropriate for the universal sequencing primer and is not limited to the example sequences described herein. Any universal sequencing primer for a specific application can be designed and used by the methods of the invention, and should not be limited to the example sequences described herein.

The released amplicons can be subjected to further amplification by the use of tail primers and secondary PCR primers. In this embodiment the primers in the droplet contain an additional sequence or tail added onto the 5′ end of the sequence specific portion of the primer. The sequences for the tailed regions are the same for each primer pair and are incorporated onto the 5′ portion of the amplicons during PCR cycling. Once the amplicons are removed from the droplets, another set of PCR primers that can hybridize to the tail regions of the amplicons can be used to amplify the products through additional rounds of PCR. The secondary primers can exactly match the tailed region in length and sequence or can themselves contain additional sequence at the 5′ ends of the tail portion of the primer. During the secondary PCR cycling these additional regions also become incorporated into the amplicons. These additional sequences can include, but are not limited to adaptor regions utilized by sequencing platforms for library preparation and sequencing, sequences used as a barcoding function for the identification of samples multiplexed into the same reaction, molecules for the separation of amplicons from the rest of the reaction materials such as biotin, digoxin, peptides, or antibodies and molecules such as fluorescent markers that can be used to identify the fragments.

### Polymerase Chain Reaction in Droplets

Methods for performing PCR in droplets are shown for example in Link et al. (U.S. patent application numbers 2008/0014589, 2008/0003142, and 2010/0137163), Anderson et al. (U.S. Pat. No. 7,041,481 and which reissued as RE41,780) and European publication number EP2047910 to Raindance Technologies Inc. The content of each of which is incorporated by reference herein in its entirety.

The sample droplet may be pre-mixed with a primer or primers, or the primer or primers may be added to the droplet. In some embodiments, droplets created by segmenting the starting sample are merged with a second set of droplets including one or more primers for the target nucleic acid in order to produce final droplets. The merging of droplets can be accomplished using, for example, one or more droplet merging techniques described for example in Link et al. (U.S. patent application numbers 2008/0014589, 2008/0003142, and 2010/0137163) and European publication number EP2047910 to Raindance Technologies Inc. In embodiments involving merging of droplets, two droplet formation modules are used.

In one embodiment, a first droplet formation module produces the sample droplets consistent with limiting or terminal dilution of target nucleic acid. A second droplet formation or reinjection module inserts droplets that contain reagents for a PCR reaction. Such droplets generally include the “PCR master mix” (known to those in the art as a mixture containing at least Taq polymerase, deoxynucleotides of type A, C, G and T, and magnesium chloride) and forward and reverse primers (known to those in the art collectively as “primers”), all suspended within an aqueous buffer. The second droplet also includes detectably labeled probes for detection of the amplified target nucleic acid, the details of which are discussed below. Different arrangements of reagents between the two droplet types is envisioned. For example, in another embodiment, the template droplets also contain the PCR master mix, but the primers and probes remain in the second droplets. Any arrangement of reagents and template DNA can be used according to the invention.

In certain embodiments, the droplet formation modules are arranged and controlled to produce an interdigitation of sample droplets and PCR reagent droplets flowing through a channel. Such an arrangement is described for example in Link et al. (U.S. patent application numbers 2008/0014589, 2008/0003142, and 2010/0137163) and European publication number EP2047910 to Raindance Technologies Inc.

A sample droplet is then caused to merge with a PCR reagent droplet, producing a droplet that includes the PCR master mix, primers, detectably labeled probes, and the target nucleic acid. Droplets may be merged for example by: producing dielectrophoretic forces on the droplets using electric field gradients and then controlling the forces to cause the droplets to merge; producing droplets of different sizes that thus travel at different velocities, which causes the droplets to merge; and producing droplets having different viscosities that thus travel at different velocities, which causes the droplets to merge with each other. Each of those techniques is further described in Link et al. (U.S. patent application numbers 2008/0014589, 2008/0003142, and 2010/0137163) and European publication number EP2047910 to Raindance Technologies Inc. Further description of producing and controlling dielectrophoretic forces on droplets to cause the droplets to merge is described in Link et al. (U.S. patent application number 2007/0003442) and European Patent Number EP2004316 to Raindance Technologies Inc. In another embodiment, called simple droplet generation, a single droplet formation module, or a plurality of droplet formation modules are arranged to produce droplets from a mixture already containing the template DNA, the PCR master mix, primers, and detectably labeled probes. In yet another embodiment, called co-flow, upstream from a single droplet formation module two channels intersect allowing two flow streams to converge. One flow stream contains one set of reagents and the template DNA, and the other contains the remaining reagents. In the preferred embodiment for co-flow, the template DNA and the PCR master mix are in one flow stream, and the primers and probes are in the other. On convergence of the flow streams in a fluidic intersection, the flow streams may or may not mix before the droplet generation nozzle. In either embodiment, some amount of fluid from the first stream, and some amount of fluid from the second stream are encapsulated within a single droplet. Following encapsulation, complete mixing occurs.

Once final droplets have been produced by any of the droplet forming embodiments above, or by any other embodiments, the droplets are thermal cycled, resulting in amplification of the target nucleic acid in each droplet. In certain embodiments, the droplets are collected off chip as an emulsion in a PCR thermal cycling tube and then thermally cycled in a conventional thermal cycler. Temperature profiles for thermal cycling can be adjusted and optimized as with any conventional DNA amplification by PCR.

In certain embodiments, the droplets are flowed through a channel in a serpentine path between heating and cooling lines to amplify the nucleic acid in the droplet. The width and depth of the channel may be adjusted to set the residence time at each temperature, which can be controlled to anywhere between less than a second and minutes.

In certain embodiments, the three temperature zones are used for the amplification reaction. The three temperature zones are controlled to result in denaturation of double stranded nucleic acid (high temperature zone), annealing of primers (low temperature zones), and amplification of single stranded nucleic acid to produce double stranded nucleic acids (intermediate temperature zones). The temperatures within these zones fall within ranges well known in the art for conducting PCR reactions. See for example, Sambrook et al. (Molecular Cloning, A Laboratory Manual, 3rd edition, Cold Spring Harbor Laboratory Press, Cold Spring Harbor, N.Y., 2001).

In certain embodiments, the three temperature zones are controlled to have temperatures as follows: 95° C. (T_{H}), 55° C. (T_{L}), 72° C. (T_{M}). The prepared sample droplets flow through the channel at a controlled rate. The sample droplets first pass the initial denaturation zone (T_{H}) before thermal cycling. The initial preheat is an extended zone to ensure that nucleic acids within the sample droplet have denatured successfully before thermal cycling. The requirement for a preheat zone and the length of denaturation time required is dependent on the chemistry being used in the reaction. The samples pass into the high temperature zone, of approximately 95° C., where the sample is first separated into single stranded DNA in a process called denaturation. The sample then flows to the low temperature, of approximately 55° C., where the hybridization process takes place, during which the primers anneal to the complementary sequences of the sample. Finally, as the sample flows through the third medium temperature, of approximately 72° C., the polymerase process occurs when the primers are extended along the single strand of DNA with a thermostable enzyme. Methods for controlling the temperature in each zone may include but are not limited to electrical resistance, peltier junction, microwave radiation, and illumination with infrared radiation.

The nucleic acids undergo the same thermal cycling and chemical reaction as the droplets passes through each thermal cycle as they flow through the channel. The total number of cycles in the device is easily altered by an extension of thermal zones or by the creation of a continuous loop structure. The sample undergoes the same thermal cycling and chemical reaction as it passes through N amplification cycles of the complete thermal device.

In other embodiments, the temperature zones are controlled to achieve two individual temperature zones for a PCR reaction. In certain embodiments, the two temperature zones are controlled to have temperatures as follows: 95° C. (T_{H}) and 60° C. (T_{L}). The sample droplet optionally flows through an initial preheat zone before entering thermal cycling. The preheat zone may be important for some chemistry for activation and also to ensure that double stranded nucleic acid in the droplets are fully denatured before the thermal cycling reaction begins. In an exemplary embodiment, the preheat dwell length results in approximately 10 minutes preheat of the droplets at the higher temperature.

The sample droplet continues into the high temperature zone, of approximately 95° C., where the sample is first separated into single stranded DNA in a process called denaturation. The sample then flows through the device to the low temperature zone, of approximately 60° C., where the hybridization process takes place, during which the primers anneal to the complementary sequences of the sample. Finally the polymerase process occurs when the primers are extended along the single strand of DNA with a thermostable enzyme. The sample undergoes the same thermal cycling and chemical reaction as it passes through each thermal cycle of the complete device. The total number of cycles in the device is easily altered by an extension of block length and tubing.

In another embodiment the droplets are created and/or merged on chip followed by their storage either on the same chip or another chip or off chip in some type of storage vessel such as a PCR tube. The chip or storage vessel containing the droplets is then cycled in its entirety to achieve the desired PCR heating and cooling cycles.

In another embodiment the droplets are collected in a chamber where the density difference between the droplets and the surrounding oil allows for the oil to be rapidly exchanged without removing the droplets. The temperature of the droplets can then be rapidly changed by exchange of the oil in the vessel for oil of a different temperature. This technique is broadly useful with two and three step temperature cycling or any other sequence of temperatures.

### Pooling

Methods of the invention further involve releasing the amplicons with attached universal sequencing primers from the droplets and pooling for further sequencing analysis. Methods of releasing amplicons from the droplets are shown in, for example, in Link et al. (U.S. patent application numbers 2008/0014589, 2008/0003142, and 2010/0137163) and European publication number EP2047910 to Raindance Technologies Inc. In certain embodiments droplets with PCR products are merged as described above.

In certain embodiments, sample droplets are allowed to cream to the top of a carrier fluid. By way of non-limiting example, the carrier fluid can include a perfluorocarbon oil that can have one or more stabilizing surfactants. The droplet rises to the top or separates from the carrier fluid by virtue of the density of the carrier fluid being greater than that of the aqueous phase that makes up the droplet. For example, the perfluorocarbon oil used in one embodiment of the methods of the invention is 1.8, compared to the density of the aqueous phase of the droplet, which is 1.0.

The creamed liquids are then placed onto a second carrier fluid which contains a destabilizing surfactant, such as a perfluorinated alcohol (e.g. 1H,1H,2H,2H-Perfluoro-1-octanol). The second carrier fluid can also be a perfluorocarbon oil. Upon mixing, the aqueous droplets begin to coalesce, and coalescence is completed by brief centrifugation at low speed (e.g., 1 minute at 2000 rpm in a microcentrifuge). The coalesced aqueous phase can now be removed and the further analyzed.

### Sequencing

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes.

A sequencing technique that can be used in the methods of the provided invention includes, for example, Helicos True Single Molecule Sequencing (tSMS) (Harris T. D. et al. (2008) Science 320:106-109). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm2. The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step. Further description of tSMS is shown for example in Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety.

Another example of a DNA sequencing technique that can be used in the methods of the provided invention is 454 sequencing (Roche) (Margulies, M et al. 2005, Nature, 437, 376-380). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used in the methods of the provided invention is SOLiD technology (Applied Biosystems). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

Another example of a DNA sequencing technique that can be used in the methods of the provided invention is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and is attached at a resolution such that the fragments are individually resolvable. Addition of one or more nucleotides releases a proton (H+), which signal detected and recorded in a sequencing instrument. The signal strength is proportional to the number of nucleotides incorporated.

Another example of a sequencing technology that can be used in the methods of the provided invention is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used in the methods of the provided invention is nanopore sequencing (Soni G V and Meller A. (2007) Clin Chem 53: 1996-2001). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used in the methods of the provided invention involves using a chemical-sensitive field effect transistor (chemFET) array to sequence DNA (for example, as described in US Patent Application Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used in the methods of the provided invention involves using an electron microscope (Moudrianakis E. N. and Beer M. Proc Natl Acad Sci USA. 1965 March; 53:564-71). In one example of the technique, individual DNA molecules are labeled using metallic labels that are distinguishable using an electron microscope. These molecules are then stretched on a flat surface and imaged using an electron microscope to measure sequences.

In a particular embodiment, the sequencing is single-molecule sequencing-by-synthesis. Single-molecule sequencing is shown for example in Lapidus et al. (U.S. Pat. No. 7,169,560), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety.

Briefly, a single-stranded nucleic acid (e.g., DNA or cDNA) is hybridized to oligonucleotides attached to a surface of a flow cell. The single-stranded nucleic acids may be captured by methods known in the art, such as those shown in Lapidus (U.S. Pat. No. 7,666,593). The oligonucleotides may be covalently attached to the surface or various attachments other than covalent linking as known to those of ordinary skill in the art may be employed. Moreover, the attachment may be indirect, e.g., via the polymerases of the invention directly or indirectly attached to the surface. The surface may be planar or otherwise, and/or may be porous or non-porous, or any other type of surface known to those of ordinary skill to be suitable for attachment. The nucleic acid is then sequenced by imaging the polymerase-mediated addition of fluorescently-labeled nucleotides incorporated into the growing strand surface oligonucleotide, at single molecule resolution.

Thus, the invention encompasses methods wherein the nucleic acid sequencing reaction comprises hybridizing a sequencing primer to a single-stranded region of a linearized amplification product, sequentially incorporating one or more nucleotides into a polynucleotide strand complementary to the region of amplified template strand to be sequenced, identifying the base present in one or more of the incorporated nucleotide(s) and thereby determining the sequence of a region of the template strand.

### Sequence Analysis and Reconstruction

For the sequence reconstruction process, short reads are stitched together bioinformatically by finding overlaps and extending them. To be able to do that unambiguously, one must ensure that long fragments that were amplified are distinct enough, and do not have similar stretches of DNA that will make assembly from short fragments ambiguous, which can occur, for example, if two molecules in a same well originated from overlapping positions on homologous chromosomes, overlapping positions of same chromosome, or genomic repeat. Such fragments can be detected during sequence assembly process by observing multiple possible ways to extend the fragment, one of which contains sequence specific to end marker. End markers can be chosen such that end marker sequence is not frequently found in DNA fragments of sample that is analyzed and probabilistic framework utilizing quality scores can be applied to decide whether a certain possible sequence extension way represents end maker and thus end of the fragment.

Overlapping fragments may be computationally discarded since they no longer represent the same initial long molecule. This process allows to treat population of molecules resulting after amplification as a clonally amplified population of disjoint molecules with no significant overlap or homology, which enables sequencing errors to be corrected to achieve very high consensus accuracy and allows unambiguous reconstruction of long fragments. If overlaps are not discarded, then one has to assume that reads may be originating from fragments originating from two homologous chromosomes or overlapping regions of the same chromosome (in case of diploid organism) which makes error correction difficult and ambiguous.

Computational removal of overlapping fragments obtained from both the 5′ and the 3′ directions also allows use of quality scores to resolve nearly-identical repeats. Resulting long fragments may be assembled into full genomes using any of the algorithms known in the art for genome sequence assembly that can utilize long reads.

In addition to de-novo assembly fragments can be used to obtain phasing (assignment to homologous copies of chromosomes) of genomic variants, by observing that under conditions of experiment described in the preferred embodiment long fragments originate from either one of chromosomes, which enables to correlate and co-localize variants detected in overlapping fragments obtained from distinct partitioned portions.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

What is claimed is:

1. A method for sequencing a nucleic acid template from both a 5′ and a 3′ end in a single sequencing reaction, the method comprising:
amplifying a nucleic acid template to produce a plurality of amplicons;
splitting the amplicons into first and second portions;
attaching a first oligonucleotide comprising a first universal primer site to a 5′ end of the amplicons in the first portion;
attaching a second oligonucleotide comprising a second universal primer site to a 3′ end of the amplicons in the second portion;
pooling the first and second portions; and
sequencing the pooled amplicons, thereby sequencing a nucleic acid template from both a 5′ and a 3′ end in a single sequencing reaction.

2. The method of claim 1, wherein amplifying is via a polymerase chain reaction.

3. The method of claim 1, wherein the 5′ and 3′ ends of each of the plurality of amplicons comprise an adaptor sequence, wherein the 5′ and the 3′ adaptor are different.

4. The method of claim 3, wherein attaching the first oligonucleotide comprises:
providing the first oligonucleotide, the oligonucleotide comprising a first portion that is complementary to the adaptor attached at the 5′ end of the amplicon and a second portion that comprises the first universal primer site; and
conducting an amplification reaction between the amplicons of the first portion and the first oligonucleotides, to thereby produce amplicons comprising the first universal primer site attached to a 5′ end of the amplicons.

5. The method of claim 4, wherein amplification is via PCR.

6. The method of claim 3, wherein attaching the second oligonucleotide comprises:
providing the second oligonucleotide, the oligonucleotide comprising a first portion that is complementary to the adaptor attached at the 3′ end of the amplicon and a second portion that comprises the second universal primer site; and
conducting an amplification reaction between the amplicons of the second portion and the second oligonucleotides, to thereby produce amplicons comprising the second universal primer site attached to a 3′ end of the amplicons.

7. The method of claim 6, wherein amplification is via PCR.

8. The method of claim 1, wherein splitting comprises compartmentalizing the amplicons into compartmentalized portions.

9. The method according to claim 8, wherein the compartmentalized portions are droplets and compartmentalizing comprises forming the droplets.

10. The method according to claim 9, wherein the droplets are aqueous droplets in an immiscible carrier fluid.

11. The method according to claim 10, wherein the immiscible carrier fluid is oil.

12. The method according to claim 11, wherein the oil comprises a surfactant.

13. The method according to claim 12, wherein the surfactant is a fluorosurfactant.

14. The method according to claim 11, wherein the oil is a fluorinated oil.

15. The method according to claim 9, wherein pooling comprises collecting the droplets in a vessel and releasing the amplicons from the droplets.

16. The method according to claim 1, wherein sequencing is sequencing-by-synthesis.

17. The method according to claim 16, wherein in sequencing-by-synthesis is single molecule sequencing-by-synthesis.

18. The method according to claim 1, wherein the first and second primer sites are different.


SEQUENCE LISTING (6 sequences)

SEQ ID NO: 1 - DNA, 17 bp, Artificial Sequence
  Synthetic sequence
cgctcttccgatctctg

SEQ ID NO: 2 - DNA, 17 bp, Artificial Sequence
  Synthetic sequence
cagtctagccttctcgt

SEQ ID NO: 3 - DNA, 47 bp, Artificial Sequence
  Synthetic sequence
ccatctcatccctgcgtgtctccgactcagcgctcttccgatctctg

SEQ ID NO: 4 - DNA, 47 bp, Artificial Sequence
  Synthetic sequence
cagtctagccttctcgtgactctgacggttccgtgtgtcccctatcc

SEQ ID NO: 5 - DNA, 47 bp, Artificial Sequence
  Synthetic sequence
cctatcccctgtgtgccttggcagtctcagcgctcttccgatctctg

SEQ ID NO: 6 - DNA, 47 bp, Artificial Sequence
  Synthetic sequence
cagtctagccttctcgtgactcagcctctgtgcgtccctactctacc